(19) United States
(12) Patent Application Publication
(10) Pub. No.: US 2016/0287482 A1
(43) Pub. Date: Oct. 6, 2016

(54) Methods and Apparatuses for Orogastric Decompression

(71) Applicant: Sarah Bassett, Laredo, TX (US)

(72) Inventor: Sarah Bassett, Laredo, TX (US)

(21) Appl. No.: 15/091,569

(22) Filed: Apr. 5, 2016

Related U.S. Application Data
(60) Provisional application No. 62/143,189, filed on Apr. 5, 2015.

(51) Int. Cl. A61J 15/00; A61M 16/04; A61M 16/00; A61M 1/00

(52) U.S. Cl. CPC A61J 15/0096; A61M 1/008; A61M 16/0415; A61M 16/0463; A61M 16/0003; A61M 2240/00

(57) ABSTRACT

A neonatal orogastric decompression device that allows simultaneous air flow and moisture absorbency. The neonatal orogastric decompression device prevents accumulation of air in the stomach and offers a more sanitary and efficient tool for health care professionals. The neonatal orogastric decompression device contains brackets in the core which hold an absorbent wedge that does not expand to block air flow from the gastrointestinal tract. The absorbent wedge has an indicator function triggered by a chemical change or accumulation of liquid. The indicator provides health care professionals with visual feedback on the quantity and quality of the liquid being absorbed.

### PRIORITY CLAIM

This application claims the benefit of the filing date of provisional application 62/143,189 filed on Apr. 5, 2015.

### FIELD OF THE DISCLOSURE

The present disclosure relates to methods and apparatuses for neonatal gastric decompression. More specifically, the present disclosure presents a hollow cylindrical syringe-like apparatus comprising a series of brackets that separate the cylindrical syringe-like apparatus into multiple compartments. One compartment allows for air to escape the neonate's stomach. One compartment allows for the absorption of fluid and secretions with an absorbent material.

### BACKGROUND OF THE DISCLOSURE

When NICU babies receive oxygen via continuous positive airway pressure (CPAP), there is a risk of air entering the stomach. When over inflation of the stomach occurs, there is a risk that the infant will vomit and aspirate gastric secretions into the lungs, causing aspiration pneumonia. Additionally, with over-inflation of the stomach, there is limited room for the diaphragm to expand, which limits lung expansion, thereby defeating the purpose of oxygen delivery. Also, air can distend the bowel, which can lead to bowel rupture.

Traditionally, to help prevent the negative effects of oxygen delivery, NICU nurses insert an orogastric (OG) tube, which is secured to the infant's face with ‘Opsite’ or whatever is used in their units. Then a 10 ml syringe is connected to the outer end of the OG tube. The nurse then folds a 2×2 inch gauze pad and fits it into the syringe. The purpose of the gauze is to absorb gastric secretions; however, when the gauze becomes saturated, it limits the escape of gastric air. When the nurses get the chance, they change either the gauze or the entire apparatus.

### SUMMARY OF THE DISCLOSURE

What is needed is a cylindrical syringe-like device with inner parts: one for liquids secretions and one for air. This way, a caregiver may no longer need to manually remove air by using an additional syringe. An orogastric decompression device for orogastric decompression has a hollow cylindrical syringe body having a plurality of brackets vertically along the inner wall of the syringe body having a distal end and a proximal end, and the brackets facing inward toward the center of the syringe body; decompression device tip; a tube to be inserted into a person's mouth and down toward the stomach; a wedge inserted into the center of the syringe and held in place by the brackets while not blocking air flow from the tube to the proximal end of the syringe; and wherein the wedge provides an indicator.

An orogastric decompression device is also included in a kit having a sterile pack and additional wedges having various indicators. An orogastric decompression device is used by inserting the tube into a neonate through the neonate's mouth; inserting the wedge through the brackets; connecting the tube to the decompression device lock on the syringe body; Positioning the syringe beside the head of the neonate to allow air flow and drainage of liquids; and monitoring liquid absorbency and saturation of the wedge.

### BRIEF DESCRIPTION OF THE DRAWINGS

The accompanying drawings, that are incorporated in and constitute a part of this specification, illustrate several embodiments of the disclosure and, together with the description, serve to explain the principles of the disclosure:

FIG. 1 illustrates an exemplary decompression device.

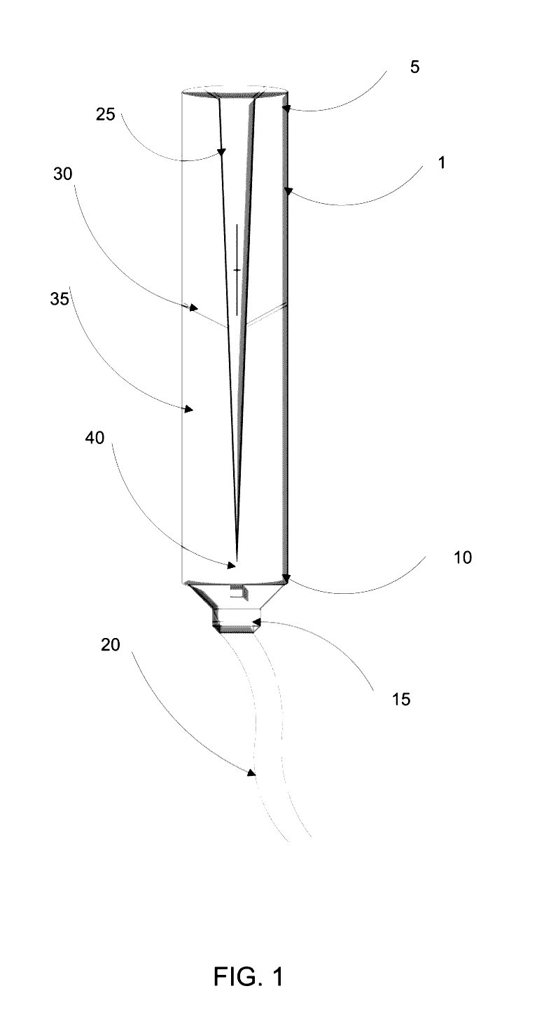

FIG. 2 illustrates an exemplary embodiment of an exemplary decompression device being used with an orogastric tube placed in a neonate.

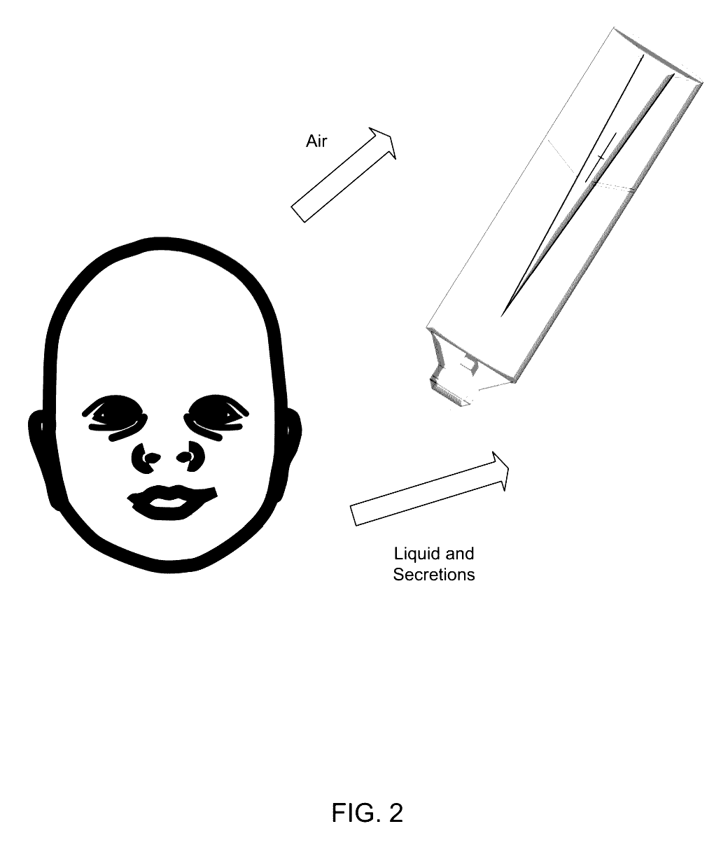

FIG. 3 illustrates an exemplary sterile pack orogastric decompression kit.

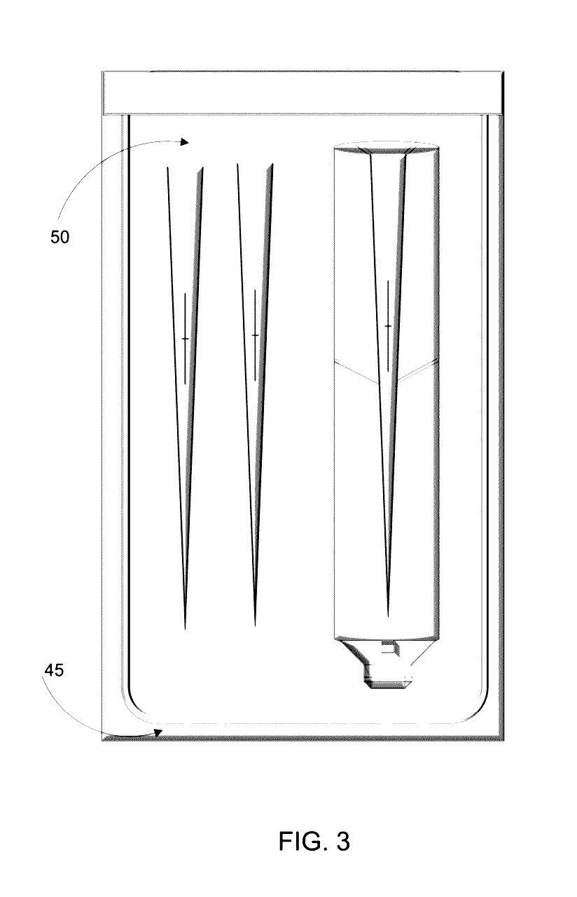

FIG. 4 illustrates an exemplary embodiment of an absorbent wedge.

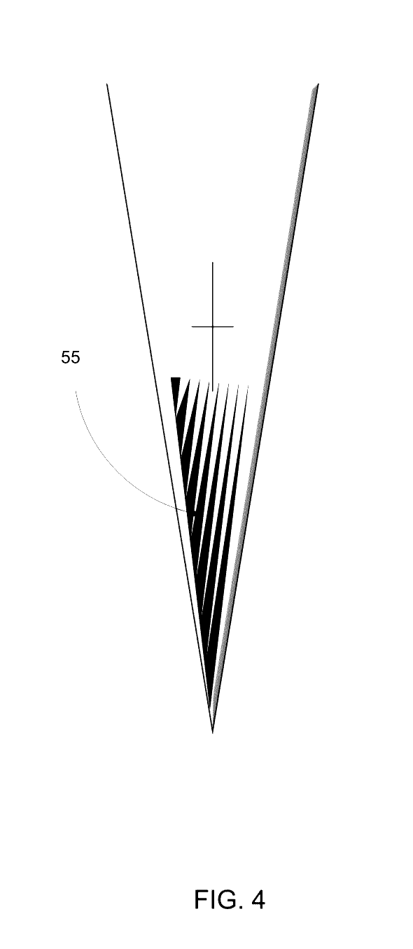

FIG. 5 illustrates exemplary method steps for using an exemplary decompression device.

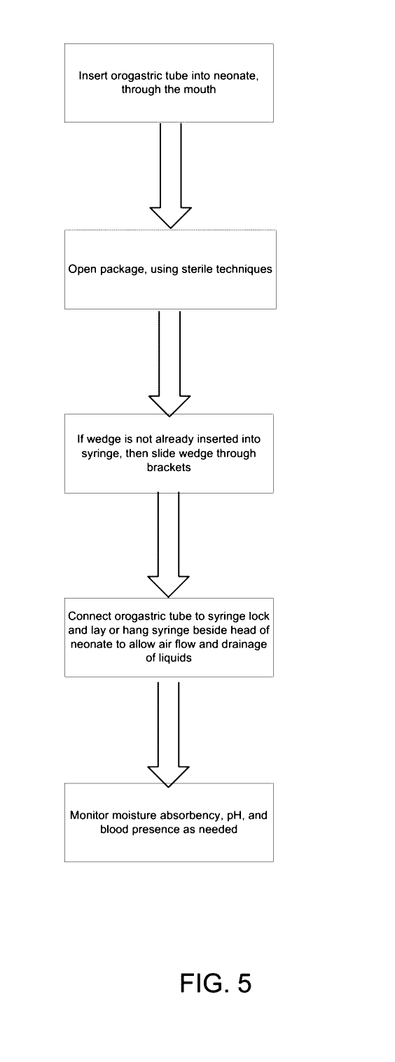

### REFERENCE NUMERALS OF THE DRAWINGS

The following list refers to the drawings:
1. Syringe Body
5. Proximal End
10. Distal End
15. Decompression Device Tip
20. Tube
25. Wedge
30. Bracket
35. Inner Wall
40. Center
45. Orogastric Decompression Kit
50. Sterile Pack
55. Indicator

### DETAILED DESCRIPTION

The present disclosure provides generally for methods and apparatuses for neonatal gastric decompression. According to the present disclosure, a neonatal gastric decompression device (“an apparatus”) comprises a cylindrical syringe, brackets inside a cylindrical syringe, and an absorbent wedge packaged in a sterile package. An apparatus may allow for air to pass through an orogastric tube and through an apparatus, thereby releasing pressure and gas in a neonate's stomach. Furthermore, an apparatus may allow for capture and storage of gastric liquids, saliva, mucous, and any other liquids or secretions.

An apparatus may be disposable, presented in a sterile package, and may also contain a plurality of spare absorbent wedges or core. Absorbent wedges may be placed by a care provider and removed by a care provider or may be permanently fixed in an apparatus. A care giver may exercise less effort in monitoring secretion absorption due to color changing properties of an absorbent wedge. Color change may be due to Litmus paper type composition or color change reaction upon contact with moisture.

In the following sections, detailed descriptions of examples and methods of the disclosure will be given. The description of both preferred and alternative examples are exemplary only, and it is understood that to those skilled in the art that variations, modifications, and alterations may be apparent. It is therefore to be understood that the examples do not limit the broadness of the aspects of the underlying disclosure as defined by the claims.

Generally, an exemplary apparatus described herein is a syringe with brackets and an absorbent wedge. An exemplary apparatus allows for a release of neonate gastric air and secretions due to gravity. An exemplary apparatus may increase productivity for caregivers, minimizes monitoring and maintenance, and reduces a buildup of gastric bloating, decreasing the need for additional devices.

### GLOSSARY

“orogastric tube” means the orogastric decompression device described herein.

“decompression device tip” means the connector part of the distal end of a syringe body.

“syringe body” means a hollow cylinder.

“bracket” means a pair of vertical protrusions in the interior of the syringe body.

“wedge” means any material suitable for absorbing liquid and having a linear shape suitable to inserting into a syringe body while not blocking air passage.

### DETAILED DESCRIPTIONS OF THE DRAWINGS

The orogastric tube described herein prevents accumulation of air in the stomach and offers a more sanitary and efficient tool for health care professionals. The orogastric tube contains brackets in the core which hold an absorbent wedge that does not expand to block air flow from the gastrointestinal tract and allows for gastric decompression. The absorbent wedge has an indicator function triggered by a chemical change or accumulation of liquid. The indicator provides health care professionals and caregivers with visual feedback on the quantity and quality of the liquid being absorbed.

In some method steps, a caregiver may insert an orogastric tube into a neonate. A caregiver may open a sterile package and connect a decompression device tip to an orogastric tube. Absorbent wedge may already be in a syringe or may be placed outside a syringe but included in a package. A package contains syringe and at least one absorbent wedge. A package may be an easy-open package. A caregiver may place an apparatus beside a neonate's head in order to facilitate drainage of liquid and capture of liquid by an absorbent core. Air may still be released rather than build up in a neonate's stomach. A caregiver or nurse may monitor an apparatus as needed. For example, a nurse may monitor every couple of hours rather than every half hour. A nurse may change an absorbent wedge or may connect a new apparatus.

An apparatus may be a 10 ml length syringe. In some embodiments, a syringe may include a tip. In some embodiments, a Luer lock or a straight lock at a tip of a syringe to a flange of a syringe may be 9 cm long and may have markings for fluid measurements. Fluid measurement markings may be single solid lines that may or may not contain measurements. Syringe may have 1.5 centimeter diameter opening/core. An apparatus may be made out of plastic. A plastic may be made out of polypropylene. A plastic may be made out of polycarbonate. In preferred embodiments, an apparatus may be transparent or clear. An apparatus may not contain a plunger. An apparatus may be individually wrapped and sterile. An apparatus may be autoclaved.

Absorbent material may check gastric placement with pH indicator, litmus paper, and/or color-changing ink in absorbent material. Once moisture contacts an absorbent wedge in a syringe, a reaction may occur that may initiate a color change. An absorbent wedge may be treated with a transitional metallic salt, such as copper chloride, to cause a color change reaction when in contact with moisture. An absorbent wedge may be treated with a halochromic material that changes color to indicate a change in pH. A single absorbent wedge may be treated with both a transitional metallic salt and a halochromic material. A caregiver may use two absorbent wedges at the same time, one to monitor pH and one to monitor moisture absorbency

A nurse or caregiver may view an absorbent wedge as needed in order to determine an approximate time to change an absorbent wedge to a new one. As a color change may occur, for example, an absorbent wedge may change from its original color of white to blue.

Absorbent wedges may be permanently fixed within a syringe. Absorbent wedges may be removeably fixed in a syringe. Brackets in a syringe may secure an absorbent wedge. Brackets may be placed multiple times along the length of a syringe. Brackets may be wide enough to secure a wet or dry absorbent wedge and prevent an absorbent wedge, wet or dry, from falling out of a syringe. A bracket should provide a snug fit. In some embodiments, brackets may be solid or mesh to allow breathability. In some embodiments, an absorbent core may be used. An absorbent core may be secured by a mesh core cage in the center of a syringe.

Absorbent material may also indicate presence of blood by turning an absorbent wedge brown due to oxidation. This may indicate a neonate may be bleeding anywhere along the alimentary canal or stomach.

Absorbent material may be comprised of natural or synthetic fibers. Preferred embodiments may be made of rayon or cotton. Wool or acrylic may also be used. In some embodiments, an absorbent wedge may include a mixture of any of the following: cotton, wool, rayon, and acrylic. Fibers may be woven tightly to create a felt-like texture. Fibers may also be compressed without being woven. Absorbency for a single absorbent wedge may range from 0 to 10 mL. A single absorbent wedge may hold 10 times its weight in liquid.

An absorbent wedge may be shorter in length than the body of a syringe. An absorbent wedge may have an isosceles triangle shape with its vertex pointed toward a syringe lock where an orogastric tube may be connected. This may maximize airflow, reduce obstruction at a syringe lock, and may allow the wicking away of moisture. A triangular shape may increase moisture-wicking surface area rather than a cylindrical shaped core having a more dense shape and lesser ability to reach and wick moisture. A wedge with a wiry texture as opposed to a smooth texture may increase its moisture wicking ability.

### CONCLUSION

A number of embodiments of the present disclosure have been described. While this specification contains many specific implementation details, the descriptions in the specification should not be construed as limitations on the scope of any disclosures or of what may be claimed, but rather as descriptions of features specific to particular embodiments of the present disclosure.

Certain features that are described in this specification in the context of separate embodiments can also be implemented in combination in a single embodiment. Conversely, various features that are described in the context of a single embodiment can also be implemented in combination in multiple embodiments separately or in any suitable sub-combination. Moreover, although features may be described above as acting in certain combinations and even initially claimed as such, one or more features from a claimed combination can in some cases be excised from the combination, and the chimed combination may be directed to a sub-combination or variation of a sub-combination.

Thus, particular embodiments of the subject matter have been described. Other embodiments are within the scope of the following claims. In some cases, the actions recited in the claims can be performed in a different order and still achieve desirable results. In addition, the processes depicted in the accompanying figures do not necessarily require the particular order show, or sequential order, to achieve desirable results. In certain implementations, multitasking and parallel processing may be advantageous. Nevertheless, it will be understood that various modifications may be made without departing from the spirit and scope of the claimed disclosure.

What is claimed is:

1. An orogastric decompression device for orogastric decompression, the device comprising
A hollow cylindrical syringe body having a plurality of brackets vertically along the inner wall of the syringe body having a distal end and a proximal end, and the brackets facing inward toward the center of the syringe body;
A decompression device tip;
A tube to be inserted into a person's mouth and down toward the stomach;
A wedge inserted into the center of the syringe and held in place by the brackets while not blocking air flow from the tube to the proximal end of the syringe; and
Wherein the wedge provides an indicator.

2. The device of claim 1 wherein the decompression device tip is a Luer lock.

3. The device of claim 1 wherein the wedge is mesh.

4. The device of claim 1 wherein the wedge is felt.

5. The device of claim 1 wherein the wedge has a pH indicator.

6. The device of claim 1 wherein the wedge has a color change indicator to indicate how saturated the wedge is with liquid.

7. The device of claim 1 wherein the wedge is fixedly attached to the inner wall of the syringe body.

8. The device of claim 1 wherein the wedge is removable.

9. The device of claim 1 wherein the indicator is halochromic.

10. An orogastric decompression kit for single use, the kit comprising
A plurality of wedges;
The orogastric decompression device of claim 1; and
A sterile pack.

11. A method of using the orogastric decompression device of claim 1, the method steps comprising
Inserting the tube into a neonate through the neonate's mouth;
Inserting the wedge through the brackets;
Connecting the tube to the decompression device lock on the syringe body;
Positioning the syringe beside the head of the neonate to allow air flow and drainage of liquids; and
Monitoring liquid absorbency and saturation of the wedge.

12. The method steps of claim 11 further comprising opening the sterile pack with sterile techniques.

13. The method steps of claim 11 further comprising removing the wedge once saturated with fluid and inserting a new wedge.
